(19) United States
(12) Patent Application Publication
(10) Pub. No.: US 2011/0040144 A1
(43) Pub. Date: Feb. 17, 2011

(54) Method and apparatus for weight control by olfactory nerve ablation

(76) Inventor: Milton S. Jackson, Louisville, KY (US)

Correspondence Address: MICHAEL J. URE, 10518 PHIL PLACE, CUPERTINO, CA, 95014, US

(21) Appl. No.: 12/856,642

(22) Filed: Aug. 14, 2010

Related U.S. Application Data
(60) Provisional application No. 61/234,385, filed on Aug. 17, 2009.

(51) Int. Cl. A61B 1/00; A61K 31/05; A61K 33/30; A61P 3/04; A61P 9/00; A61P 3/06; A61P 3/10; A61P 19/00; A61P 25/28

(52) U.S. Cl. 600/104; 514/731; 424/643

(57) ABSTRACT

Methods and apparatus are described for diet and weight loss control by altering the senses of smell and taste. Olfactory nerves are ablated in a localized area at the top of the nasal cavity, resulting in a loss of smell, which also decreases the sense of taste and results in a reduced appetite, enabling a patient to eat less and lose weight. After several months the nerves begin to regenerate, and the sense of smell and taste return, although a repeat ablation can be performed if necessary, such as if new dietary habits have not yet been firmly established.

### RELATED APPLICATION

This application claims priority of U.S. application Ser. No. 61/234,385 of the present inventor filed Aug. 17, 2009, entitled “Treatment for Obesity: Ablation of the Olfactory Nerve Blocks Taste,” incorporated herein by reference.

### BACKGROUND

Obesity is a major healthcare problem with approximately 64 percent of American adults being overweight. Half of these people are considered morbidly obese. Many children and teenagers also struggle with weight. Obesity is related to hypertension, arthritis, chronic pain, diabetes, heart disease, strokes, many cancers, pulmonary diseases and respiratory failure, and many other diseases and disabilities. Obesity costs Americans tens of billions of dollars annually, significantly shortens people's life spans, and affects quality of life.

A person's weight is the end result of the relationship of food and eating compared to growth, metabolism, and exercise.

Taste is a combination of sensations of sweet, sour, salt, bitter, and texture on the tongue and the sense of smell. The sense of smell is the most important component of taste. Taste helps trigger appetite and hunger.

### SUMMARY

The present invention is defined by the following claims, and nothing in this section should be taken as a limitation on those claims. Methods are described for aiding diet and weight control by blocking a person's sense of smell. Without an associated smell, most foods are significantly less appealing.

The olfactory nerve enters the superior aspect of the nasal cavity through the cribriform plate. The nerve's branches spread out over the top of the inner aspect of the nose. Receptors on the olfactory nerve are able to differentiate about 10,000 odors.

Ablation of the olfactory nerve at the level of the cribriform plate blocks the transmission of neural sensations to the brain. Therefore, odors are not appreciated by the brain.

According to one embodiment of the present invention, a chemo-ablation is applied to the olfactory nerve at the location of the cribriform plate. Nerve ablation is achieved through direct application of a chemical or desiccant, such as phenol or zinc sulfate. Although the nerve is desiccated, the nerve sheath is not destroyed. The olfactory nerves typically regenerate over a period of four to six months.

Ablation of the olfactory nerve at the cribriform plate blocks neural transmission to the brain by destroying the olfactory nerve cells. The cells are then no longer able to function. Smell is no longer sensed, and food becomes relatively tasteless, except for the relatively simple tastes sensed by the tongue. The relatively tasteless food is generally unappealing. A person eats less food and weight loss is achieved. Studies have shown that animals will also eat less if their food has no taste. New eating habits are developed. Although there is some risk of permanent nerve damage, a person will typically not be able to smell or have normal taste for a period of four to six months. A patient under a physician's care who is trying to lose weight will find food less appealing, will eat less, and will be more effective in losing weight.

Medicinal blockage of olfaction, such as through the use of nose sprays, are temporary, and are associated with cost issues, compliance issues, and have side-effects. Chemo-ablation of the olfactory nerve is semi-permanent, inexpensive, and has no side-effect.

### BRIEF DESCRIPTION OF THE DRAWINGS

The present invention may be further understood from the following description in conjunction with the appended drawings. In the drawings:

FIG. 1 is an illustration of nasal cavity.

FIG. 2 is a flowchart illustrating the inclusion of olfactory ablation in weight loss management.

### DETAILED DESCRIPTION

Described herein are methods and apparatus for inclusion in a weight loss program of an overweight patient.

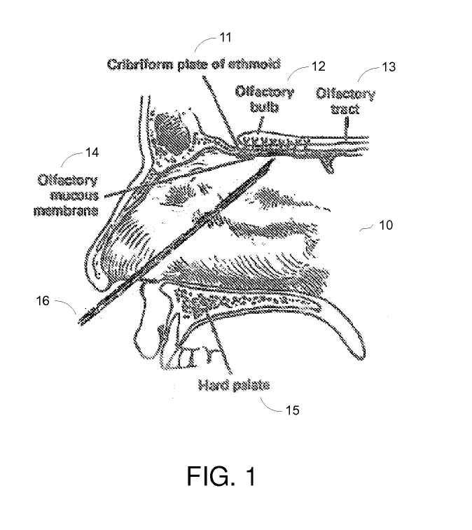

FIG. 1 shows patient anatomy, consisting of a nasal cavity (10), a cribriform plate of ethmoid (11), an olfactory bulb (12), an olfactory tract (13), olfactory mucous membrane (14), and a hard palate (15). In the roof, or superior portion of the nasal cavity (10), is the cribriform plate of ethmoid (11), also commonly called simply the cribriform plate. It lies laterally on either side of a medial ridge, the crista galli, which is not shown. Above, or superior to the cribriform plate is the olfactory bulb (12), which lies at end of the olfactory tract (13).

The olfactory nerve, not shown, passes through or along the olfactory tract (13) to the region of the olfactory bulb (12), where it descends through holes, or fenestrations, in the cribriform plate (11). The olfactory nerve then spreads out throughout the tissue below the cribriform plate, largely along the upper, or superior portion of the nasal cavity (10). The olfactory mucous membrane (14) provides an external barrier to the air. The hard palate (15) is a bony structure in the floor, or inferior portion of the nasal cavity. Also shown is a needle (16) through which an injection can be made into the olfactory nerve in the vicinity of the cribriform plate.

The extent of the olfactory nerve, or nerves, can vary considerably from patient to patient with unreliable boundaries, as they spread out across the nasal cavity. Therefore, it may be difficult to construct a physical device that will reliably encapsulate or block the receptors of all of the olfactory nerves. However, because all of the olfactory nerves pass through the cribriform plate, injections of neurotoxins in this area can effectively eliminate function of all of the olfactory nerves. Depending on the dose and the neurotoxin, a particular range of ablation efficacy can be established for each injection. Multiple injections in the relatively small region of the cribriform plate can then be made to ensure compete overlap, thus ablating or generating an interruption in the communicating path of all olfactory nerves, resulting in a cessation of a patient's sense of smell.

As previously noted, although the olfactory nerve is ablated, or desiccated, and thus ceases to function, the nerve sheath is not destroyed, and the olfactory nerve will regenerate, typically over a period of several months.

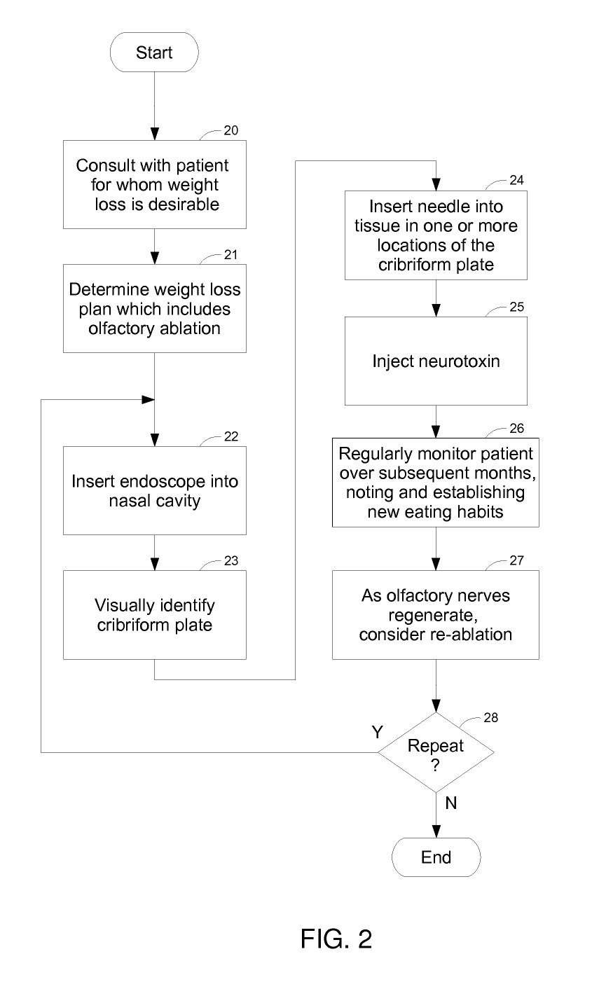

FIG. 2 shows a flowchart that incorporates olfactory ablation in the management of a patient. Initially a physician consults with a patient for whom weight loss is desirable (20). There are a large number of reasons why it may be desirable for a patient to lose weight. The patient may have a clinical condition that is complicated or made worse by excessive patient weight. The patient may be at risk of developing a clinical condition for which excessive weight is a risk factor. The patient may simply desire an improved quality of life associated with less weight. The patient may or may not have previously tried lifestyle or interventional changes in an attempt to lose weight.

After consulting with the patient, the physician determines a weight loss plan for the patient. Such a plan may incorporate a particular diet, some amount of increased exercise, the support of a coach or support group, or alternatively or in conjunction with these or other known or to be developed weight loss techniques, the inclusion of olfactory nerve ablation (21).

The olfactory ablation is performed by inserting a endoscope into the nasal cavity (22). The endoscope contains an optical or visual system that allows the physician to see into the nasal cavity. It may include a light source. With this optical or visual system, the physician identifies the cribriform plate (23). This is a relatively small region, roughly the size of a dime or less than 2 cm in diameter, through which all olfactory nerves pass.

A needle is then inserted through the olfactory mucous membrane and into the tissue in this region (24). The needle may be passed through a port in the endoscope, a needle may be passed into the nasal cavity in another way. A neurotoxin, such as phenol or zinc sulfate, is injected (25) through the needle into the tissue. Phenol and zinc sulfate are known neurotoxins, with known dosages for their efficacious use. When properly used, they will desiccate or effectively destroy the targeted nerves, but they do not have unintended physiological side effects. Multiple injections can be quickly made throughout the region, if necessary, to confidently ablate or interrupt the path of all of the olfactory nerves.

With the olfactory ablation, the patient experiences a cessation in his or her sense of smell. A lack of smell has been found to result in a reduced desire to eat. Combined with a motivation to eat less, a patient may be more successful in dieting. The physician should regularly monitor the patient over the subsequent months (26), as the patient establishes new eating habits. Without a sense of smell, the patient may experience a change in the types of food that he or she desires, and care should be taken that the newly desired foods are healthy and conducive to the dieting process.

The olfactory nerves regenerate over a course of several months, and with that regeneration comes a return of the patient's sense of smell. The physician should consider whether another olfactory ablation would be in the patient's best interest (27). If the ablation is proving effective and the patient might benefit from another ablation, then the procedure may be repeated (28, 22). If the patient has lost sufficient weight and has firmly established new dietary eating habits, a subsequent ablation may not be necessary. If the ablation has not had any affect on the patient's weight loss, for whatever reason, the physician may elect to not repeat the procedure, and the patient will, over time, regain their sense of smell.

While the invention has been described above by reference to various embodiments, it will be understood that many changes and modifications can be made without departing from the scope of the invention. For example, the neuro-toxin injecting needle may be incorporated into the endoscope, or may be distinct from the endoscope but still under visual guidance. Additionally, neurotoxins other than those described herein, which may be currently known or not yet known, may be used.

It is therefore intended that the foregoing detailed description be understood as an illustration of the presently preferred embodiments of the invention, and not as a definition of the invention. It is only the following claims, including all equivalents that are intended to define the scope of the invention.

What is claimed is:

1. A method for diet and weight control of a subject through chemo-ablation of an olfactory nerve, the method comprising the step of injecting a drug into a region of the olfactory nerve whereby the function of the olfactory nerve is suppressed.

2. The method of claim 1, wherein the subject suffers from a condition from the group consisting of obesity and a physiological disorder associated with obesity.

3. The method of claim 2, wherein the physiological disorder associated with obesity is a member of the group consisting of atherosclerosis, coronary artery disease, hypertension, hypercholesterolemia, diabetes mellitus, gout, osteoarthritis, intertrigo, sleep apnea, hyperlipidemia, hypolipidemia, hypoproteinemia, Pickwickian syndrome, and Cushing syndrome.

4. The method of claim 1, wherein the drug suppresses olfactory nerve function by blocking olfactory neurotransmission.

5. The method of claim 1, wherein the drug is a neurotoxin.

6. The method of claim 5, wherein the neurotoxin is from the group of phenol and zinc sulfate.

7. The method of claim 5, further comprising a subsequent injection, wherein the subsequent injection occurs after the olfactory nerves have at least partially regenerated.

8. The method of claim 7, wherein the subsequent injection is within an interval of 2 to 9 months after the previous injection.

9. The method of claim 1, wherein the injecting further comprises the steps of:
a) inserting an endoscope into the nasal cavity;
b) identifying a cribriform plate; and
c) injecting the drug into a region of the cribriform plate.

10. The method of claim 9, wherein the injected drug is a neurotoxin.

11. The method of claim 10, wherein the neurotoxin is from the group of phenol and zinc sulfate.

12. An apparatus for injecting a neurotoxin into a region of a cribriform plate, comprising:
a) an endoscope with an optical portion for identifying the cribriform plate, and
b) a needle, the needle being capable to be positioned, under optical guidance, into a viscinity of the cribriform plate and capable of a injecting neurotoxin into olfactory nerves in the region of the cribriform plate.

13. A method of appetite suppression comprising:
identifying a patient in need of weight loss; and
injecting the patient to dull the patient's sense of smell so as to suppress the patient's appetite.
